(19) United States
(12) Patent Application Publication
(10) Pub. No.: US 2026/0108213 A1
(43) Pub. Date: Apr. 23, 2026

(54) MULTI-STAGE CARDIAC ACTIVITY EVALUATION

(71) Applicant: Cardiac Pacemakers, Inc., St Paul, MN (US)

(72) Inventors: Jonathan H. Kelly, Woodbury, MN (US); Derek D. Bohn, Woodbury, MN (US)

(21) Appl. No.: 19/363,038

(22) Filed: Oct. 20, 2025

Related U.S. Application Data
(60) Provisional application No. 63/709,715, filed on Oct. 21, 2024.

(51) Int. Cl. A61B 5/00; A61B 5/024; A61B 5/0245; A61B 5/29; A61B 5/318; G06N 20/00

(52) U.S. Cl. CPC A61B 5/7264; A61B 5/02416; A61B 5/0245; A61B 5/29; A61B 5/318; G06N 20/00

(57) ABSTRACT

Systems and methods include approaches involving detecting potential cardiac events using a rule-based algorithm and based on cardiac data that is time-series cardiac data, identifying first sections of the cardiac data that are associated with the potential cardiac events, identifying second sections of the cardiac data that are not associated with any of the potential cardiac events, and classifying, using an inferential machine learning model, the potential cardiac events contained in the first sections into a first set of different types of cardiac rhythms.

### TECHNICAL FIELD

This application claims priority to Provisional Application No. 63/709,715, filed Oct. 21, 2024, which is herein incorporated by reference in its entirety.

### TECHNICAL FIELD

The present disclosure generally relates to multi-stage approaches for evaluating cardiac activity.

### BACKGROUND

Medical devices that allow physicians to monitor cardiac activity are becoming increasingly common in diagnosing and treating medical conditions in patients. Cardiac monitoring can be used, for example, to identify abnormal cardiac rhythms, so that critical alerts can be provided to patients, physicians, or other care providers and so that patients can be treated as needed.

### SUMMARY

In Example 1, a method includes detecting potential cardiac events using a rule-based algorithm and based on cardiac data that is time-series cardiac data, identifying first sections of the cardiac data that are associated with the potential cardiac events, identifying second sections of the cardiac data that are not associated with any of the potential cardiac events, and classifying, using an inferential machine learning model, the potential cardiac events contained in the first sections into a first set of different types of cardiac rhythms.

In Example 2, the method of Example 1, further including classifying, using the rule-based algorithm, the potential cardiac events into a second set of different types of cardiac rhythms.

In Example 3, the method of Example 2, wherein the classifying the potential cardiac events into the first set of different types of cardiac rhythms is based, at least in part, on the second set of different types of cardiac rhythms inputted into the inferential machine learning model.

In Example 4, the method of any of Examples 1-3, wherein only the first sections of the cardiac data are inputted into the inferential machine learning model, wherein the second sections of the cardiac data are not inputted into the inferential machine learning model.

In Example 5, the method of any of Examples 1-4, wherein the rule-based algorithm and the inferential machine learning model are operated on a server.

In Example 6, the method of any of Examples 1-4, wherein the rule-based algorithm is operated on a patient device, wherein the inferential machine learning model is operated on a server.

In Example 7, the method of Example 6, wherein the patient device is an implantable medical device, a monitoring device attached to a patient, or a mobile computing device.

In Example 8, the method of any of Examples 1-7, wherein the rule-based algorithm comprises thresholds that are compared to the cardiac data, wherein the inferential machine learning model comprises a trained neural network.

In Example 9, the method of Example 8, wherein the thresholds include heart rate thresholds and a duration threshold.

In Example 10, the method of Example 9, wherein the heart rate thresholds include a minimum heart rate and a maximum heart rate, wherein the duration threshold includes a minimum duration threshold.

In Example 11, the method of any of Examples 1-10, wherein the cardiac data is continuously and wirelessly transmitted from a patient device to an intermediate device, wherein the intermediate device wirelessly transmits the cardiac data to a server.

In Example 12, the method of any of Examples 1-11, wherein the cardiac data is electrocardiogram data or photoplethysmography data.

In Example 13, a computer program product comprising instructions to cause one or more processors to carry out the steps of the method of Examples 1-12.

In Example 14, a computer-readable medium having stored thereon the computer program product of Example 13.

In Example 15, a server comprising the computer-readable medium of Example 14.

In Example 16, a system includes a server and one or more processors. The one or more processors are programmed to operate instructions to cause the system to: detect potential cardiac events using a rule-based algorithm and based on cardiac data that is time-series cardiac data, identify first sections of the cardiac data that are associated with the potential cardiac events, and identifying second sections of the cardiac data that are not associated with any of the potential cardiac events. The server is configured to operate a machine learning model to classify the potential cardiac events contained in the first sections into a first set of different types of cardiac rhythms.

In Example 17, the system of Example 16, wherein only the first sections of the cardiac data are inputted into the machine learning model, wherein the second sections of the cardiac data are not inputted into the machine learning model.

In Example 18, the system of Example 16, wherein the server comprises the one or more processors.

In Example 19, the system of Example 16, further comprising a mobile computing device comprising the one or more processors.

In Example 20, the system of Example 16, further including a medical device comprising electrodes and configured to generate the time-series cardiac data.

In Example 21, the system of Example 20, wherein the medical device comprises the one or more processors.

In Example 22, the system of Example 20, wherein the medical device is an implantable medical device.

In Example 23, the system of Example 16, wherein the rule-based algorithm comprises thresholds that are compared to the cardiac data.

In Example 24, the system of Example 23, wherein the thresholds include heart rate thresholds and a duration threshold.

In Example 25, the system of Example 24, wherein the heart rate thresholds include a minimum heart rate and a maximum heart rate, wherein the duration threshold includes a minimum duration threshold.

In Example 26, the system of Example 16, wherein the cardiac data is electrocardiogram data or photoplethysmography data.

In Example 27, the system of Example 16, wherein the cardiac data is continuously and wirelessly received from a patient device to an intermediate device, wherein the intermediate device is programmed to wirelessly transmit the cardiac data to the server.

In Example 28, a method includes detecting potential cardiac events using a rule-based algorithm and based on cardiac data that is time-series cardiac data, identifying first sections of the cardiac data that are associated with the potential cardiac events, identifying second sections of the cardiac data that are not associated with any of the potential cardiac events, and classifying, using an inferential machine learning model, the potential cardiac events contained in the first sections into a first set of different types of cardiac rhythms.

In Example 29, the method of Example 28, wherein only the first sections of the cardiac data are inputted into the inferential machine learning model, wherein the second sections of the cardiac data are not inputted into the inferential machine learning model.

In Example 30, the method of Example 28, wherein the rule-based algorithm and the inferential machine learning model are operated on a server.

In Example 31, the method of Example 28, wherein the rule-based algorithm is operated on a patient device, wherein the inferential machine learning model is operated on a server.

In Example 32, the method of Example 28, wherein the rule-based algorithm comprises thresholds that are compared to the cardiac data, wherein the inferential machine learning model comprises a trained neural network.

In Example 33, the method of Example 32, wherein the thresholds include heart rate thresholds and a duration threshold.

In Example 34, the method of Example 33, wherein the heart rate thresholds include a minimum heart rate and a maximum heart rate, wherein the duration threshold includes a minimum duration threshold.

In Example 35, the method of Example 28, wherein the cardiac data is continuously and wirelessly transmitted from a patient device to an intermediate device, wherein the intermediate device wirelessly transmits the cardiac data to a server.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIGS. 1 and 2 are schematic illustrations of a cardiac event evaluation system, in accordance with certain instances of the present disclosure.

FIG. 3 shows a block diagram depicting an illustrative method, in accordance with certain instances of the disclosure.

FIG. 4 shows a schematic of a graphical user interface, in accordance with certain instances of the disclosure.

FIG. 5 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Monitoring devices can be equipped with one or more sensing components (e.g., sensors, electrodes) and programmed to sense physiological data such as electrocardiogram (ECG) data and/or photoplethysmography (PPG) data. To collect physiological data, one or more devices (e.g., implantable cardiac monitors/recorders, external cardiac monitors/recorders) can be implanted in or coupled to the patient such that the devices can sense data. For example, a physician may initiate a study during which a monitoring device that senses physiological data of a patient.

To detect and classify cardiac events, the physiological data can be inputted into a machine learning model such as a neural network (e.g., a trained neural network, a trained deep learning neural network). The machine learning model can process the physiological data and output data such as rhythm classifications, beat classifications, heart rates, etc.

Processing physiological data with a machine learning model takes time, and operating machine learning models can consume a significant amount of power. Because a patient may experience few cardiac events during a study, it can be inefficient to process the entire study using a machine learning model. For example, only 5% or less (e.g., 4% or less, 3% or less) of cardiac activity during a 24-hour period may contain cardiac data associated with a potential cardiac events—with the rest of the cardiac data containing normal cardiac activity.

Certain instances of the present disclosure are accordingly directed to approaches that use multiple stages to process and evaluate physiological data. In certain instances, a first stage utilizes a rule-based evaluation and a subsequent stage utilizes an inference-based evaluation.

### Cardiac Event Evaluation System

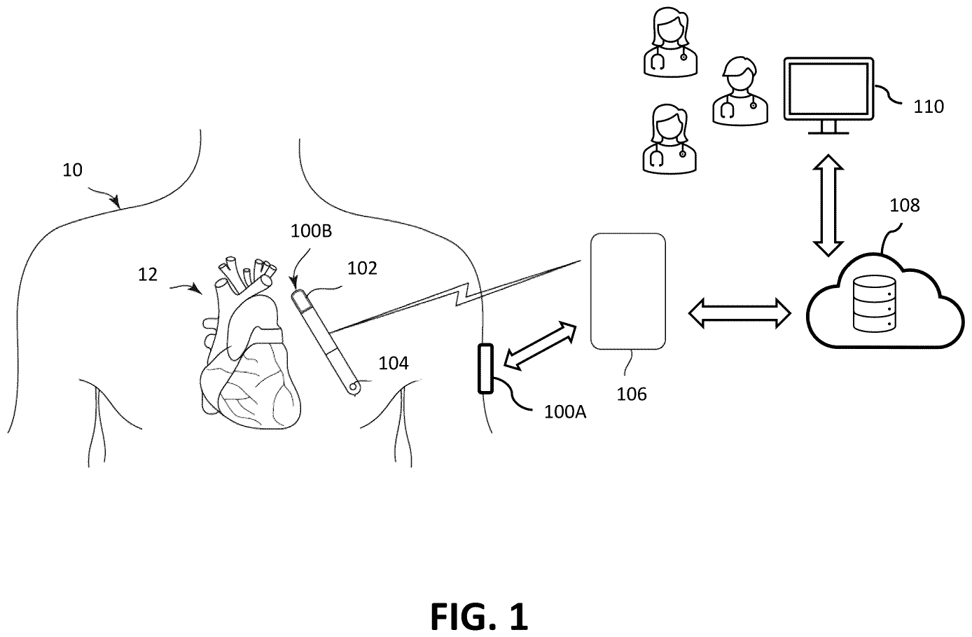

FIG. 1 is a schematic illustration of a cardiac event evaluation system that includes an external device 100A such as a monitoring device (e.g., patch, watch, ring) that is coupled to a patient 10 such as their skin or a mobile device that includes one or more electrodes and/or sensors (e.g., optical sensor) for detecting physiological data. The physiological data can include ECG data and/or PPG data. ECG data can include time-series data such as ECG waveforms that represent sensed cardiac electric activity over time. PPG data can include time-series data that indicates, over time, a person's blood oxygen content.

Additionally or alternatively, the system can include an implantable medical device 100B. The implantable medical device 100B can be implanted subcutaneously within an implantation location or pocket in the patient's chest or abdomen and may be configured to sense physiological signals associated with the patient's heart 12. The implantable medical device 100B may be an implantable cardiac monitor (e.g., an implantable diagnostic monitor, an implantable loop recorder) configured and programmed to record physiological data such as, for example, one or more cardiac activation signals, heart sounds, blood pressure measurements, oxygen saturations. Although the implantable medical device 100B of FIG. 1 is shown as an implantable cardiac monitor, the approaches described herein can be used in connection with other types of implantable medical devices such as a pulse generator (e.g., pacemaker, defibrillator).

In the example of FIG. 1, the implantable medical device 100B includes electrodes 102 and 104, which are used to sense physiological signals (e.g., electrical cardiac activation signals) of the heart 12 and generate ECG data. Additionally or alternatively, the implantable medical device 100B could include an optical sensor (e.g., a light sensor) for generating PPG data.

As described in more detail below, certain physiological data can be communicated (e.g., communicated wirelessly using an antenna of the devices 100A/100B) to a different component within the system. For example, physiological data can be communicated from the medical device 100 to a receiver 106, a computing system 108, and/or a remote computing device 110. The receiver 106 can be a device that is capable of programming, controlling, monitoring, and/or otherwise communicating with the devices 100A and 100B. The receiver 106 can help facilitate communication from the device 100A and 100B to another device or system such as the computing system 108 (e.g., laptop computer, desktop computer, server). The receiver 106 and/or the computing system 108 can be communicatively coupled to another computing system 110 with a display on which users (e.g., patients, physicians, technicians) can view data sensed and recorded by the medical device 100. In certain instances, the receiver 106 is a mobile computing device such as a programmer, smartphone, tablet computer, laptop computer, etc.

The devices 100A and 100B can be programmed to initially store sensed physiological data to local memory (e.g., cache, buffer). However, local memory can have limited storage capacity due to power and/or space constraints. To deal with limited storage capacity, the devices 100A and 100B can be programmed to cause physiological data to be transmitted to the receiver 106 and/or the computing system 108. The transmitted physiological data can then be deleted from the local memory to allow new physiological data to be stored to the local memory. The process of storing sensed physiological data to local memory, transmitting it to a different device, deleting the transmitted physiological data from the local memory, and then storing newly-sensed physiological data to the local memory can be repeated throughout a study or monitoring period.

In certain instances, the monitoring device being used during a study can be programmed to continuously transmit the sensed physiological data to a separate device such as the patient's smartphone or another receiver device. In other instances or certain situations, the monitoring device may transmit chunks or strips of a predetermined length of time or file size to a separate device. The receiver device can be programmed to then transmit the received physiological data to a server or other computing device. The receiver device can, likewise, continuously transmit the sensed physiological data or in discrete chunks.

As noted above, processing physiological data with a machine learning model takes time, and operating machine learning models can consume a significant amount of power.

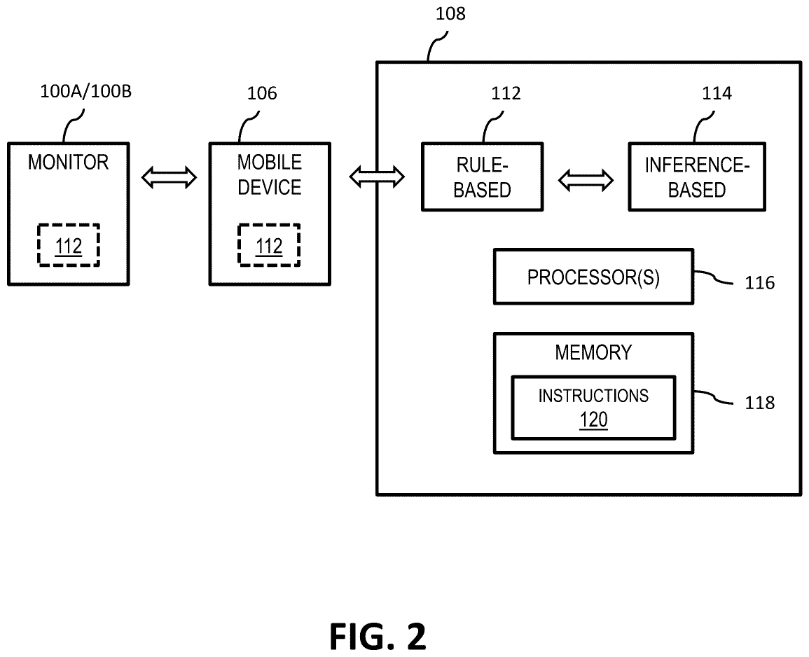

FIG. 2 shows a block diagram of a multi-stage architecture for processing and evaluating physiological data. The architecture described herein can help reduce the amount of energy used to process and evaluate physiological data compared to approaches that process all physiological data of a study with a machine learning model. In short, an initial stage of the architecture can use a rule-based evaluation approach to identify potential cardiac events, and a subsequent stage of the architecture can use an inference-based evaluation approach to process the potential cardiac events. In some instances, the rule-based evaluation approach does not use a machine learning model, whereas the inference-based evaluation approach uses a machine learning model. As such, the rule-based evaluation approach can be more power-efficient than the inference-based evaluation approach.

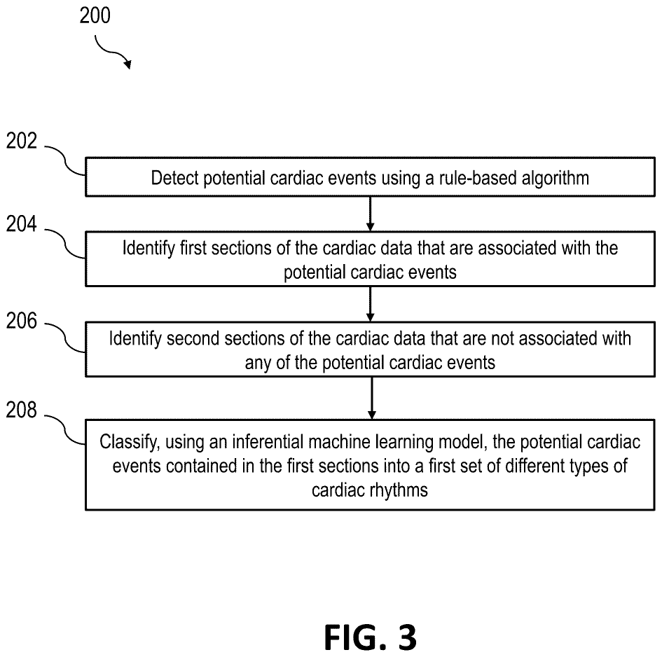

In FIG. 2, the computing system 108 includes a rule-based module 112 and an inference-based module 114. The computing system 108 can include one or more processors 116 along with memory 118 and computer-readable instructions or code 120. Each module can include a block of the computer-readable code or instructions 120 such that each module is programmed to carry out one or more of the functions described herein. Various functions of the multi-stage architecture of FIG. 2 are described in connection with FIG. 3.

FIG. 3 shows a block diagram of a method 200. The method 200 includes detecting potential cardiac events using one or more rule-based algorithms (block 202 in FIG. 3) such as one or more algorithms carried out by the rule-based module 112 of FIG. 2. The potential cardiac events are detected based on physiological data (e.g., physiological data representing a person's cardiac activity) inputted to the rule-based algorithm. The physiological data can be ECG data or PPG data—both of which can be considered to be time-series cardiac data.

The potential cardiac events can be detected using rules such as one or more thresholds set to identify cardiac events like bradycardia, tachycardia, pause, atrial tachycardia, atrial fibrillation, premature ventricular contraction (PVC), among others. For example, potential bradycardia events can be based on a person's heart rate dropping below a threshold (e.g., 60 bpm, 50 bpm, 40 bpm, 30 bpm) for a certain period of time (e.g., 1 second, 2 seconds, 3 seconds, 4 seconds). As another example, potential tachycardia events can be based on a person's heart rate rising above a threshold (e.g., 140 bpm, 150 bpm, 160 bpm, 170 bpm) for a certain period of time (e.g., 3 seconds, 4seconds, 5 seconds, 6 seconds, 7 seconds). As another example, potential pause events can be based on a beat-to-beat time rising above a threshold (e.g., 2 seconds, 3, seconds, 4 seconds, 5 seconds). As another example, potential atrial tachycardia events can be based on a person's average heart rate rising above a threshold (e.g., 140 bpm, 150 bpm, 160 bpm, 170 bpm) over a certain period of time (e.g., 1 hour, 2 hours, 3 hours, 4 hours, 5hours). As another example, potential atrial fibrillation events and PVC events can be based on detecting that such events have occurred more than a threshold number of times over a period of time.

As noted above, at least some of the thresholds are based on a person's heart rate. As such, in some instances, the time-series cardiac data is used to calculate heart rates, etc., which are inputted into the one or more rule-based algorithms. For example, one of the devices 100A/100B may calculate heart rates, etc., and then transmit the calculated heart rates to the computing system 108. In other instances, the rule-based module 112 itself is programmed to calculated desired heart rates, etc.

In certain instances, the rules or thresholds used by the one or more rule-based algorithms are system-level or global thresholds such that the same rules or thresholds are used for all patients. In these instances, the rules or thresholds can be set to be more sensitive to potential cardiac events to err on the side of detecting false-positive events rather than missing actual cardiac events.

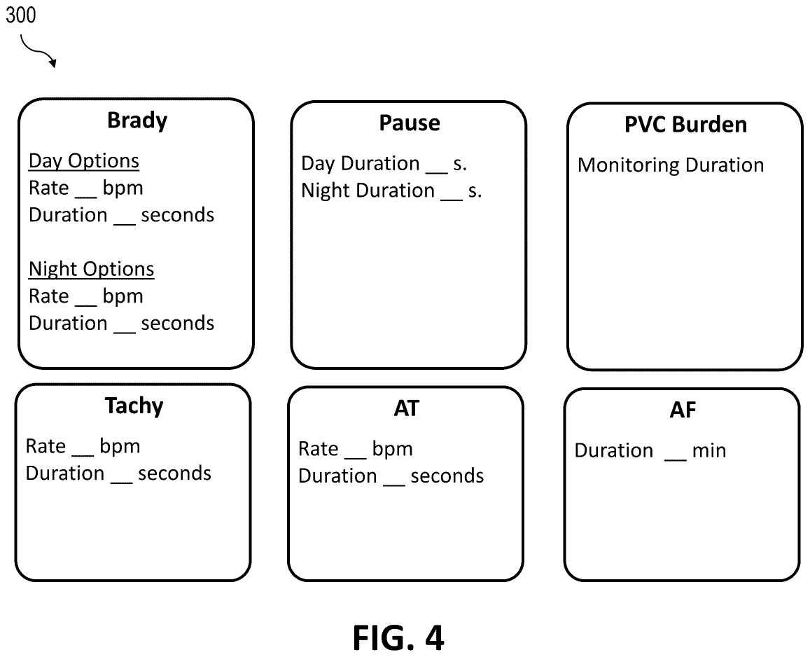

In other instances, one or more rules or thresholds can be customized on a patient-by-patient basis. FIG. 4 shows a schematic of an example graphical user interface 300 (hereinafter the “interface 300” for brevity) that can be used to customize one or more rules or thresholds. For example, the interface 300 can be used to change thresholds for heart rates, durations, event numbers, and the like. The thresholds can also be set differently for different parts of a 24-hour period (e.g., daytime settings versus nighttime settings). The interface 300 can also be used to ignore certain types of potential cardiac events. For example, if a person's physician was mostly concerned with fast heart rates, the physician could customize the rule-based algorithms to only detect potential cardiac events associated with fast heart rates as opposed to low heart rates. The interface 300 can have various screens with icons, drop-down menus, input cells, etc., so that one can use the interface to adjust settings related to thresholds, alerts, etc.

Referring back to FIG. 3, the method 200 includes identifying first sections of the cardiac data that are associated with the potential cardiac events (block 204 in FIG. 3) and identifying second sections of the cardiac data that are not associated with any of the potential cardiac events (block 206 in FIG. 3). Put another way, after the potential cardiac events have been detected, the underlying physiological data can be split into sections that include at least one of the potential cardiac events and sections that do not include at least one of the potential cardiac events. Each section can be identified by a start time and end time, and each section can be assigned a unique label (e.g., unique alphanumerical value) so that each section can be separately identified.

In certain instances, once a potential cardiac event is detected, a predetermined amount of physiological data before the onset of the potential cardiac event (e.g., 1 minute or less, 30 seconds or less, 15 seconds or less) and a predetermined amount of physiological data after the end of the potential cardiac event (e.g., 1 minute or less, 30 seconds or less, 15 seconds or less) can be compiled into the first sections of the cardiac data. Physiological data not associated with such sections can be grouped into the second sections (e.g., strips of physiological data that do not contain potential cardiac events detected by one or more of the rule-based algorithms).

In some instances, in addition to identifying the sections that include at least one of the potential cardiac events, each potential cardiac event can be associated with a classification (e.g., identifying the potential cardiac event as a tachycardia event, bradycardia event, and the like). For example, the rule-based module 112 can be programmed to not only detect potential cardiac events, but to also label or associate each potential cardiac event with a classification such as tachycardia, bradycardia, pause, etc., as described herein. As such, the rule-based module 112 can generate metadata (e.g., rhythm classification) and associate the metadata to respective sections of the physiological data.

The method 200 further includes classifying, using a machine learning model (e.g., an inferential machine learning model), the potential cardiac events contained in the first sections into a first set of different types of cardiac rhythms (block 208 in FIG. 3). The machine learning model can process the physiological data associated with the potential cardiac events as well as, in certain instances, metadata generated by the rule-based algorithms such as the initial classifications.

Because a person may experience few potential cardiac events during a study or may experience potential cardiac events that are spread out in time, it can be inefficient to process the entire study using a machine learning model. To save power and computing resources (compared to if the entire study was processed by the machine learning model), only the sections of physiological data that include potential cardiac events (as detected by the one or more rule-based algorithms) can be inputted and processed by the machine learning model.

Referring back to FIG. 2, the inference-based module 114 can include one or more machine learning models to process the physiological data and classify different cardiac events. In certain instances, the machine learning model(s) include one or more different types of deep neural networks. In classifying the cardiac events, the machine learning model may compare the physiological data to labeled physiological data to determine which labeled physiological data the physiological data most closely resembles. The comparison of the physiological data can include comparing amplitudes of the ECG waveform or the PPG waveform at different points in time with amplitudes of labeled (e.g., classified) ECG or PPG waveforms. The labeled physiological data may identify a particular cardiac event such as those described herein.

In certain instances, the machine learning model includes two paths, where the first path is a deep convolutional neural network and the second path is a deep fully-connected neural network. The deep convolutional neural network receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, and a non-linear activation function layer. This set of layers can be repeated multiple times.

The deep fully connected neural network can receive RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

If the machine learning model determines that the physiological data most closely resembles a labeled physiological data associated with a cardiac event, then the machine learning model may determine that the patient has experienced that cardiac event. Additionally, the machine learning model may measure or determine certain characteristics of the cardiac activity of the patient based on the physiological data. For example, the machine learning model may determine a heart rate, a duration, and/or a beat count of the patient during the cardiac event based on the physiological data. The computing system 108 stores the cardiac event and associated metadata such as information like beat classification (e.g., normal, ventricular, supraventricular, unknown), heart rate, duration, beat count, etc., in a database for storage.

In some instances, the rule-based module 112 is operated by the computing system 108 such as a server. In other instances, the rule-based module 112 can be operated by the devices 100A or 100B. In other instances, the rule-based module 112 can be operated by the receiver 106 such as a mobile computing device like a smart phone. Similarly, the inference-based module 114 could be operated by different devices or systems other than a server.

Regardless of which device or system operates the rule-based module 112, the physiological data can be continuously and wirelessly transmitted from the devices 100A or 100B to an intermediate device (e.g., the receiver 106), and the intermediate device can wirelessly transmit the physiological data to the computing system 108 for additional processing.

Using the architecture of FIG. 2, physiological data can be initially processed using a first stage of the architecture to detect potential health events. This first stage acts as an initial screen or filter to identify periods of physiological data without potential health events. As noted herein, the first stage can be carried out by various devices within a system (e.g., monitoring device, intermediate device, computing system).

The remaining physiological data can be processed using a second stage of the architecture of FIG. 2 such as an inferential approach like the machine learning model(s) described herein. Because the physiological data has been initially screened or filtered, the amount of physiological data processed using the inferential approach is reduced—which can save time and computing powered required to process physiological data of a study. In instances, where the first stage outputs a classification of the potential cardiac events, the second stage can confirm or change the initial classification.

### Computing Devices and Systems

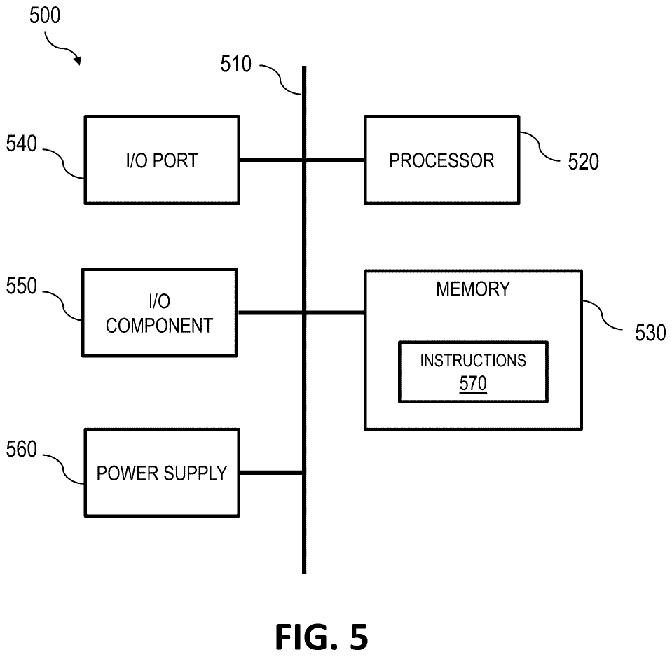

FIG. 5 is a block diagram depicting an illustrative computing device 500, in accordance with instances of the disclosure. The computing device 500 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 5 and described below. For example, the monitoring devices, receivers, and computing systems can each include their own set (or partial set) of components shown in FIG. 5 and described below.

In instances, the computing device 500 includes a bus 510 that, directly and/or indirectly, couples one or more of the following devices: a processor 520, a memory 530, an input/output (I/O) port 540, an I/O component 550, and a power supply 560. Any number of additional components, different components, and/or combinations of components may also be included in the computing device 500.

The bus 510 represents what may be one or more busses (such as, for example, an address bus, data bus, or combination thereof). Similarly, in instances, the computing device 500 may include a number of processors 520, a number of memory components 530, a number of I/O ports 540, a number of I/O components 550, and/or a number of power supplies 560. Additionally, any number of these components, or combinations thereof, may be distributed and/or duplicated across a number of computing devices.

In instances, the memory 530 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 530 stores computer-executable instructions 570 for causing the processor 520 to implement aspects of instances of components discussed herein and/or to perform aspects of instances of methods and procedures discussed herein. The memory 530 can comprise a non-transitory computer readable medium storin the computer-executable instructions 570.

The computer-executable instructions 570 may include, for example, computer code, machine-useable instructions, and the like such as, for example, program components capable of being executed by one or more processors 520 (e.g., microprocessors) associated with the computing device 500. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 570 may be configured to be executed by the processor 520 and, upon execution, to cause the processor 520 to perform certain processes. In certain instances, the processor 520, memory 530, and instructions 570 are part of a controller such as an application specific integrated circuit (ASIC), field-programmable gate array (FPGA), and/or the like. Such devices can be used to carry out the functions and steps described herein.

The I/O component 550 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
one or more processors programmed to operate instructions to cause the system to:
detect potential cardiac events using a rule-based algorithm and based on cardiac data that is time-series cardiac data,
identify first sections of the cardiac data that are associated with the potential cardiac events, and
identifying second sections of the cardiac data that are not associated with any of the potential cardiac events; and
a server configured to operate a machine learning model to classify the potential cardiac events contained in the first sections into a first set of different types of cardiac rhythms.

2. The system of claim 1, wherein only the first sections of the cardiac data are inputted into the machine learning model, wherein the second sections of the cardiac data are not inputted into the machine learning model.

3. The system of claim 1, wherein the server comprises the one or more processors.

4. The system of claim 1, further comprising a mobile computing device comprising the one or more processors.

5. The system of claim 1, further comprising:
a medical device comprising electrodes and configured to generate the time-series cardiac data.

6. The system of claim 5, wherein the medical device comprises the one or more processors.

7. The system of claim 5, wherein the medical device is an implantable medical device.

8. The system of claim 1, wherein the rule-based algorithm comprises thresholds that are compared to the cardiac data.

9. The system of claim 8, wherein the thresholds include heart rate thresholds and a duration threshold.

10. The system of claim 9, wherein the heart rate thresholds include a minimum heart rate and a maximum heart rate, wherein the duration threshold includes a minimum duration threshold.

11. The system of claim 1, wherein the cardiac data is electrocardiogram data or photoplethysmography data.

12. The system of claim 1, wherein the cardiac data is continuously and wirelessly received from a patient device to an intermediate device, wherein the intermediate device is programmed to wirelessly transmit the cardiac data to the server.

13. A method comprising:
detecting potential cardiac events using a rule-based algorithm and based on cardiac data that is time-series cardiac data;
identifying first sections of the cardiac data that are associated with the potential cardiac events;
identifying second sections of the cardiac data that are not associated with any of the potential cardiac events; and
classifying, using an inferential machine learning model, the potential cardiac events contained in the first sections into a first set of different types of cardiac rhythms.

14. The method of claim 13, wherein only the first sections of the cardiac data are inputted into the inferential machine learning model, wherein the second sections of the cardiac data are not inputted into the inferential machine learning model.

15. The method of claim 13, wherein the rule-based algorithm and the inferential machine learning model are operated on a server.

16. The method of claim 13, wherein the rule-based algorithm is operated on a patient device, wherein the inferential machine learning model is operated on a server.

17. The method of claim 13, wherein the rule-based algorithm comprises thresholds that are compared to the cardiac data, wherein the inferential machine learning model comprises a trained neural network.

18. The method of claim 17, wherein the thresholds include heart rate thresholds and a duration threshold.

19. The method of claim 18, wherein the heart rate thresholds include a minimum heart rate and a maximum heart rate, wherein the duration threshold includes a minimum duration threshold.

20. The method of claim 13, wherein the cardiac data is continuously and wirelessly transmitted from a patient device to an intermediate device, wherein the intermediate device wirelessly transmits the cardiac data to a server.
